(19) United States
(12) Patent Application Publication
(10) Pub. No.: US 2025/0292889 A1
(43) Pub. Date: Sep. 18, 2025

(54) IMAGE DISTRIBUTION SYSTEM, IMAGE DISTRIBUTION METHOD, AND IMAGE DISTRIBUTION DEVICE

(71) Applicant: Takayuki Inoue, KANAGAWA (JP)

(72) Inventor: Takayuki Inoue, KANAGAWA (JP)

(73) Assignee: Ricoh Company, Ltd., Tokyo (JP)

(21) Appl. No.: 19/051,619

(22) Filed: Feb. 12, 2025

(30) Foreign Application Priority Data
Mar. 13, 2024 (JP) 2024-038728

(51) Int. Cl. G16H 30/20; G06F 3/01

(52) U.S. Cl. CPC G16H 30/20; G06F 3/013

(57) ABSTRACT

An image distribution system includes a first terminal device and an information processing device. The first terminal device is operated by a viewer. The information processing device controls distribution of a captured image of a subject user to the first terminal device. The information processing device includes circuitry that manages information on the viewer permitted to view the captured image of the subject user, distributes the captured image of the subject user to the first terminal device of the managed viewer, and registers the viewer as the managed viewer in response to an action of the subject user to register the viewer. The first terminal device includes a display to display the distributed captured image of the subject user.

### CROSS-REFERENCE TO RELATED APPLICATION

This patent application is based on and claims priority pursuant to 35 U.S.C. § 119(a) to Japanese Patent Application No. 2024-038728, filed on Mar. 13, 2024, in the Japan Patent Office, the entire disclosure of which is hereby incorporated by reference herein.

### BACKGROUND

### Technical Field

The present disclosure relates to an image distribution system, an image distribution method, and an image distribution device.

### Related Art

Image distribution techniques have been proposed that distribute images of a patient under care to an application installed in a tablet terminal or a smartphone of a healthcare worker or a family member at a remote place.

### SUMMARY

The present disclosure described herein provides an image distribution system including a first terminal device and an information processing device. The first terminal device is operated by a viewer. The information processing device controls distribution of a captured image of a subject user to the first terminal device. The information processing device includes circuitry that manages information on the viewer permitted to view the captured image of the subject user, distributes the captured image of the subject user to the first terminal device of the managed viewer, and register the viewer as the managed viewer in response to an action of the subject user to register the viewer. The first terminal device includes a display to display the distributed captured image of the subject user.

The present disclosure described herein provides an image distribution method including managing information on a viewer permitted to view a captured image of a subject user, distributing the captured image of the subject user to a first terminal device of the managed viewer, registering the viewer as the managed viewer in response to an action of the subject user to register the viewer, and displaying the distributed captured image of the subject user.

The present disclosure described herein provides an image distribution device including circuitry. The circuitry manages information on a viewer permitted to view a captured image of a subject user, distributes the captured image of the subject user to a first terminal device of the managed viewer, and register the viewer as the managed viewer in response to an action of the subject user to register the viewer.

### BRIEF DESCRIPTION OF THE DRAWINGS

A more complete appreciation of embodiments of the present disclosure and many of the attendant advantages and features thereof can be readily obtained and understood from the following detailed description with reference to the accompanying drawings, wherein:

FIG. 1 is a diagram illustrating an image distribution system;

FIG. 2 is a block diagram illustrating a hardware configuration of a smartphone;

FIG. 3 is a block diagram illustrating a hardware configuration of a computer;

FIG. 4 is a block diagram illustrating a hardware configuration of a spherical image capturing device;

FIG. 5 is a diagram illustrating an overview of an image distribution system;

FIG. 6 is a block diagram illustrating a functional configuration of an image distribution system;

FIG. 7 is a sequence diagram illustrating a process of setting viewers in an image distribution system;

FIG. 8 is a table of information on subject users;

FIG. 9 is a table of viewer candidate data;

FIGS. 10A, 10B, and 10C are tables associated with a team identification (ID), a family ID, and a group ID, respectively;

FIG. 11 is an image diagram illustrating a screen of a viewing management application;

FIG. 12 is a sequence diagram illustrating a process of setting viewers in an image distribution system;

FIG. 13 is a sequence diagram illustrating a process of setting a viewer in response to a request from the viewer in an image distribution system;

FIG. 14 is a sequence diagram illustrating a process of setting a viewer in response to a request from the viewer in an image distribution system;

FIG. 15 is a sequence diagram illustrating a process of controlling a distribution destination of captured images of a subject user to reflect the intention of the subject user in an image distribution system;

FIG. 16 is a table of use cases of an image distribution system; and

FIG. 17 is a sequence diagram illustrating a process of requesting a viewer to be set to take a photograph.

The accompanying drawings are intended to depict embodiments of the present disclosure and should not be interpreted to limit the scope thereof. The accompanying drawings are not to be considered as drawn to scale unless explicitly noted. Also, identical or similar reference numerals designate identical or similar components throughout the several views.

### DETAILED DESCRIPTION

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

Referring now to the drawings, embodiments of the present disclosure are described below.

As used herein, the singular forms “a,” “an,” and “the” are intended to include the plural forms as well, unless the context clearly indicates otherwise. The term “connected/coupled” includes both direct connections and connections in which there are one or more intermediate connecting elements.

For the sake of simplicity, identical or similar reference numerals denote identical or similar elements such as parts and materials having the same functions, and redundant descriptions thereof are omitted unless otherwise required.

### System Configuration

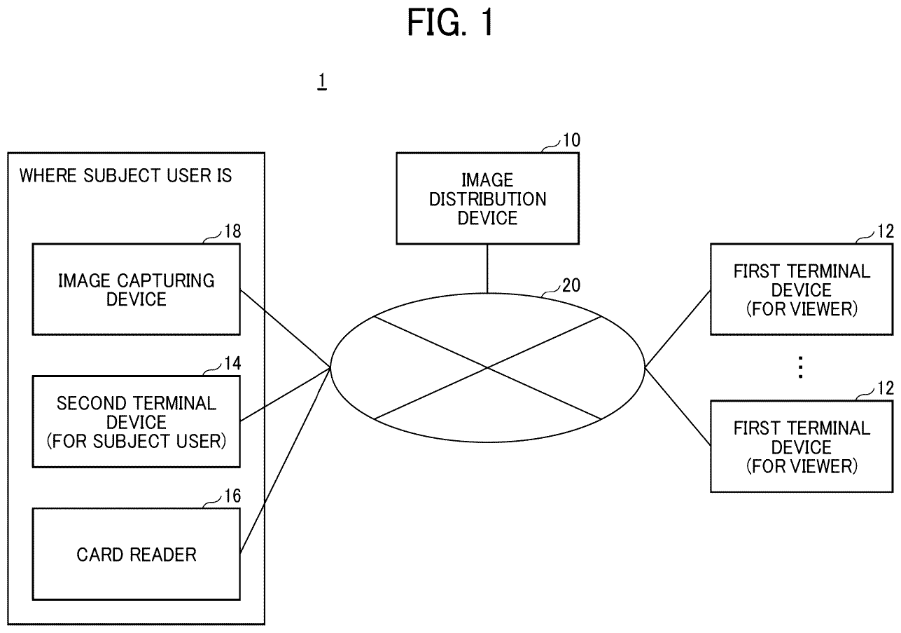

FIG. 1 is a diagram illustrating an image distribution system 1.

The image distribution system 1 illustrated in FIG. 1 includes an image distribution device 10, a first terminal device 12, a second terminal device 14, a card reader 16, and an image capturing device 18.

The image distribution device 10, the first terminal device 12, the second terminal device 14, the card reader 16, and the image capturing device 18 are connected to each other through a network 20 to be able to communicate with each other.

The network 20 is, for example, the Internet or a local area network (LAN).

The second terminal device 14, the card reader 16, and the image capturing device 18 are present where the subject user is.

The subject user is a user who is a subject of images distributed by the image distribution system 1. The subject user is, for example, a patient under care and may be at a hospital, the home of the patient under care, or a nursing facility.

The image capturing device 18 is an example of a device that captures images (photographs and videos) of a subject user and transmits the captured images (photographs and videos) to the image distribution device 10.

The image capturing device 18 having the function of communicating with the network 20 and transmits the captured images to the image distribution device 10 through the network 20.

The image capturing device 18 that does not have the function of communicating with the network 20 may be connected to the network 20 through a communication terminal having the function of communicating with the network 20 and transmit the captured images to the image distribution device 10 through the communication terminal and the network 20.

The image capturing device 18 may be a communication terminal such as a smartphone having an image capturing function.

The image capturing device 18 may be a wide-angle camera such as a spherical image capturing device, or the image capturing device 18 does not have to be a wide-angle camera.

As a spherical image capturing device, for example, a digital camera that captures a 360-degree image in one shot may be used.

The 360-degree image is merely an example and may be a panoramic image obtained by capturing a wide area.

The second terminal device 14 or the card reader 16 is an example of a device that receives an action of a subject user.

The image distribution system 1 includes at least one of the second terminal device 14 and the card reader 16.

The second terminal device 14 is an example of a device operated by a subject user.

The second terminal device 14 is, for example, a mobile phone, a smartphone, a tablet terminal, a personal computer (PC), a game console, or a personal digital assistant (PDA).

The second terminal device 14 receives, as an action of the subject user, an instruction from the subject user through manual input, voice input, or line-of-sight input.

The card reader 16 is an example of a device that reads information from a recording medium such as a magnetic card or an integrated circuit (IC) card.

In the present embodiment, the recording medium such as a magnetic card or an IC card is, for example, an Individual Number Card or a patient registration card of the subject user.

The card reader 16 receives, as an action of the subject user, an instruction to read information from a recording medium associated with the information on the viewer.

The first terminal device 12 is an example of a device operated by a viewer.

The viewer is a user who views the images of the subject user distributed by the image distribution system 1 and is somewhere other than where the subject user is.

The first terminal device 12 is, for example, a mobile phone, a smartphone, a tablet terminal, a PC, a game console, or a PDA.

The image distribution device 10 receives the images of the subject user captured by the image capturing device 18.

The image distribution device 10 manages viewers (specifically, the information on viewers) who are permitted to view the images of the subject user captured by the image capturing device 18. The image distribution device 10 distributes the images of the subject user captured by the image capturing device 18 to the first terminal device 12 of the viewer who is permitted to view the images.

The image distribution device 10 may distribute and reproduce the images of the subject user captured by the image capturing device 18 to the first terminal device 12 of the viewer by live streaming through the network 20.

When the image of the subject user captured by the image capturing device 18 is a 360-degree image, the viewer can select the angle of view of the image of the subject user reproduced by the first terminal device 12 and view the image at a free angle for each viewer.

When the second terminal device 14 or the card reader 16 receives an action of the subject user as described later, the image distribution device 10 controls the distribution destination of the images of the subject user to reflect the intention of the subject user.

The image distribution device 10 is, for example, a PC or a workstation.

The image distribution device 10 may be implemented by a server, an application service provider (ASP), or cloud computing.

The image distribution system 1 controls the distribution destination of the images of the subject user to reflect the intention of the subject user and thus can distribute the private information of the subject user in an appropriate range and prevent the private information from being distributed to an unintended user.

The configuration of the image distribution system 1 illustrated in FIG. 1 is merely an example.

For example, another device may have at least some of the functions of the image distribution device 10.

The image distribution device 10 may be a multifunction peripheral (MFP), which is an example of an image forming apparatus.

Other examples of the image distribution device 10 include, but are not limited to, a projector (PJ), an interactive whiteboard (IWB; a whiteboard having an electronic blackboard function), an output device such as digital signage, an industrial machine, an imaging device, a sound collecting device, a medical device, a networked home appliance, and a connected car.

### Hardware Configuration

### Smartphone

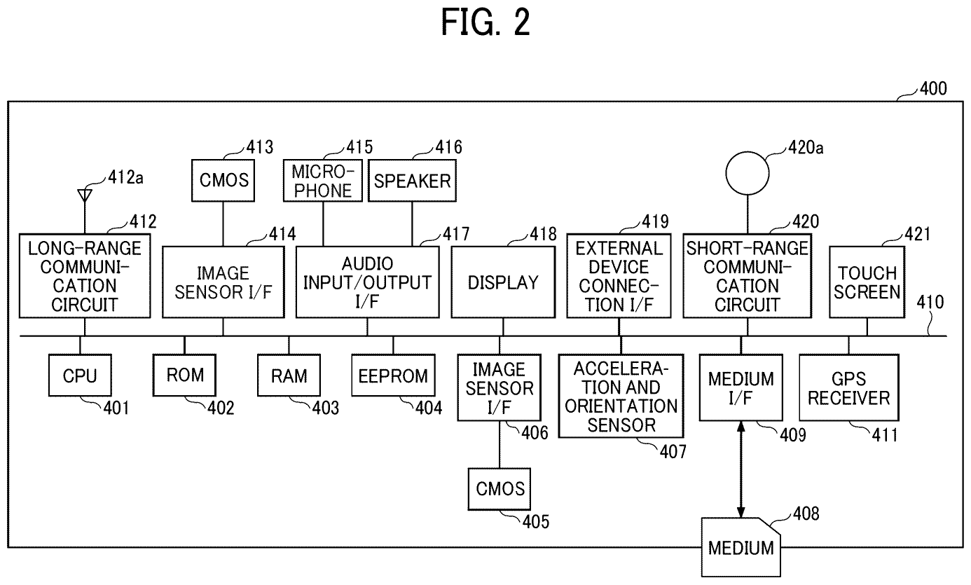

Each of the first terminal device 12 and the second terminal device 14 is implemented by, for example, a smartphone 400 having a hardware configuration illustrated in FIG. 2.

FIG. 2 is a block diagram illustrating a hardware configuration of the smartphone 400.

The smartphone 400 includes a central processing unit (CPU) 401, a read-only memory (ROM) 402, a random-access memory (RAM) 403, an electrically erasable programmable read-only memory (EEPROM) 404, a complementary metal oxide semiconductor (CMOS) sensor 405, an image sensor interface (I/F) 406, an acceleration and orientation sensor 407, a medium I/F 409, and a Global Positioning System (GPS) receiver 411.

The CPU 401 controls the overall operation of the smartphone 400.

The ROM 402 stores a program such as an initial program loader (IPL) to boot the CPU 401.

The RAM 403 is used as a work area for the CPU 401.

The EEPROM 404 reads or writes various data such as a control program for the smartphone under the control of the CPU 401.

The CMOS sensor 405 is an example of built-in imaging means that captures an image of a subject (e.g., a self-image of a user) under the control of the CPU 401 to obtain image data.

In alternative to the CMOS sensor 405, imaging means such as a charge-coupled device (CCD) sensor may be used.

The image sensor I/F 406 is a circuit that controls the driving of the CMOS sensor 405.

The acceleration and orientation sensor 407 includes various sensors such as an electromagnetic compass for detecting geomagnetism, a gyrocompass, and an acceleration sensor.

The medium I/F 409 controls the reading or writing (storing) of data from or to a recording medium 408 such as a flash memory.

The GPS receiver 411 receives a GPS signal from a GPS satellite.

The smartphone 400 further includes a long-range communication circuit 412, an antenna 412a for the long-range communication circuit 412, a CMOS sensor 413, an image sensor I/F 414, a microphone 415, a speaker 416, an audio input/output I/F 417, a display 418, an external device connection I/F 419, a short-range communication circuit 420, an antenna 420a for the short-range communication circuit 420, and a touch screen 421.

The long-range communication circuit 412 is a circuit that allows the smartphone 400 to communicate with other devices through the network 20.

The CMOS sensor 413 is an example of built-in imaging means that captures an image of a subject under the control of the CPU 401 to obtain image data.

The image sensor I/F 414 is a circuit that controls the driving of the CMOS sensor 413.

The microphone 415 is a built-in circuit that converts sound into electrical signals.

The speaker 416 is a built-in circuit that converts electrical signals into physical vibrations to generate sound such as music or voice.

The audio input/output I/F 417 is a circuit for inputting or outputting an audio signal between the microphone 415 and the speaker 416 under the control of the CPU 401.

The display 418 is an example of display means that displays, for example, an image of a subject and various icons. The display 418 may be, for example, a liquid crystal display (LCD) or an organic electroluminescence display (OELD).

The external device connection I/F 419 is an interface for connecting the smartphone 400 to various external devices.

The short-range communication circuit 420 is a communication circuit in compliance with, for example, near field communication (NFC) or BLUETOOTH.

The touch screen 421 is an example of input means that enables a user to operate the smartphone 400 by touching or pressing a screen of the display 418.

The smartphone 400 further includes a bus line 410.

The bus line 410 is, for example, an address bus or a data bus that electrically connects the elements illustrated in FIG. 2, such as the CPU 401, with one another.

### Computer

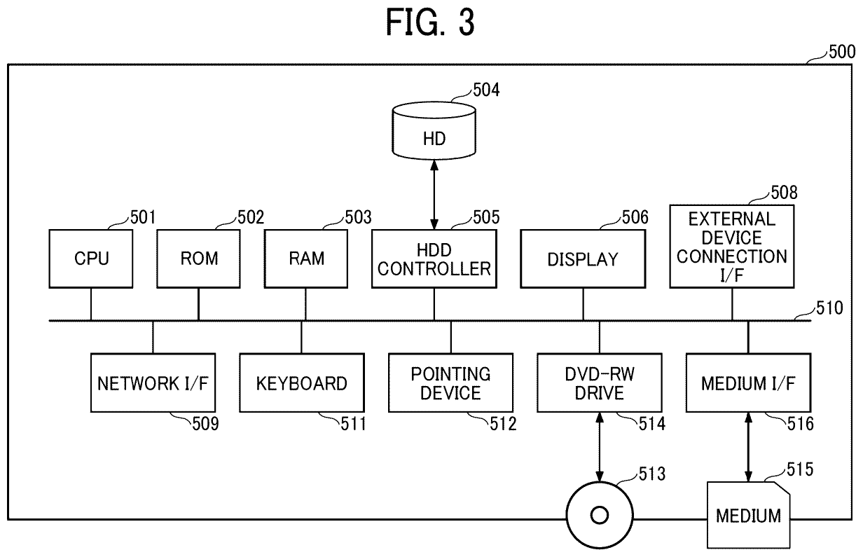

The image distribution device 10 is implemented by, for example, a computer 500 having a hardware configuration illustrated in FIG. 3.

Each of the first terminal device 12 and the second terminal device 14 may be implemented by the computer 500 having a hardware configuration illustrated in FIG. 3.

FIG. 3 is a block diagram illustrating a hardware configuration of the computer 500.

The computer 500 includes a CPU 501, a ROM 502, a RAM 503, a hard disk (HD) 504, a hard disk drive (HDD) controller 505, a display 506, an external device connection I/F 508, a network I/F 509, a data bus 510, a keyboard 511, a pointing device 512, a digital versatile disk-rewritable (DVD-RW) drive 514, and a medium I/F 516.

The CPU 501 controls the overall operation of the computer 500 according to a program.

The ROM 502 stores programs such as an IPL to boot the CPU 501.

The RAM 503 is used as a work area for the CPU 501.

The HD 504 stores various data such as a program.

The HDD controller 505 controls the reading or writing of various data from or to the HD 504 under the control of the CPU 501.

The display 506 displays various types of information such as a cursor, menu, window, character, or image.

The display 506 may be, for example, a head-mounted display (HMD) or a head up display (HUD).

The external device connection I/F 508 is an interface for connecting the computer 500 to various external devices, such as a universal serial bus (USB) flash drive.

The network I/F 509 is an interface that enables data communication through the network 20.

The data bus 510 is, for example, an address bus or a data bus that electrically connects the elements such as the CPU 501 with one another.

The keyboard 511 is an example of input means including multiple keys that allow a user to input characters, numerals, or various instructions.

The pointing device 512 is an example of input means that allows a user to, for example, select or execute a specific instruction, select a target for processing, or move a cursor being displayed.

The DVD-RW drive 514 controls the reading or writing of various data from or to a DVD-RW 513, which is an example of a removable recording medium.

The removable recording medium is not limited to the DVD-RW. For example, the removable recording medium may be a DVD-recordable (DVD-R).

The medium I/F 516 controls the reading or writing (storing) of data from or to a recording medium 515 such as a flash memory.

The hardware configuration illustrated in FIG. 3 is merely an example. The computer 500 may not include some of the elements illustrated in FIG. 3, or the computer 500 may include one or more elements other than those illustrated in FIG. 3.

### Spherical Image Capturing Device

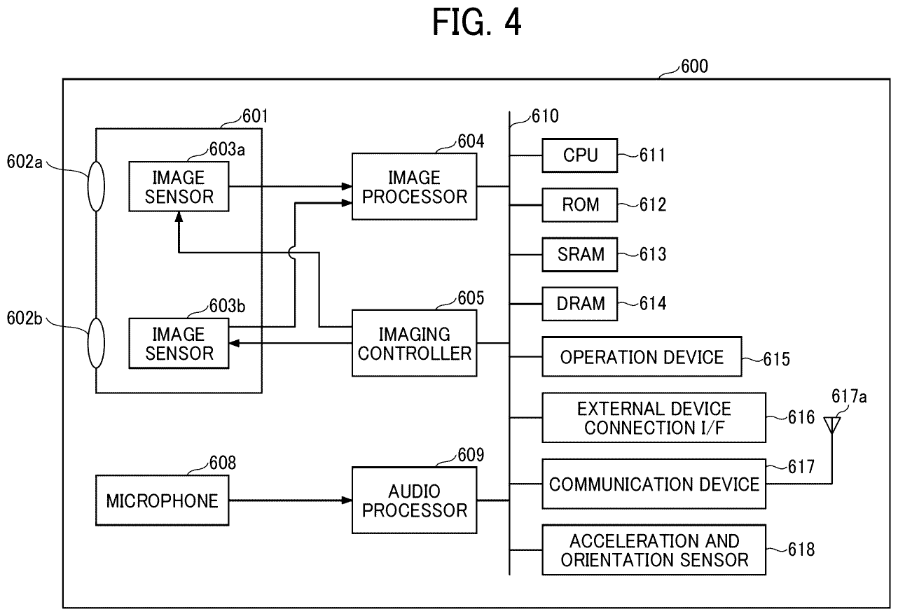

The image capturing device 18 is implemented by, for example, a spherical image capturing device 600 having a hardware configuration illustrated in FIG. 4.

FIG. 4 is a block diagram illustrating a hardware configuration of the spherical image capturing device 600.

The spherical image capturing device 600 illustrated in FIG. 4 is an example of a spherical (omnidirectional) image capturing device with two image sensors. Alternatively, the spherical image capturing device 600 may include more than two image sensors.

The spherical image capturing device 600 does not have to be a device dedicated to spherical image capturing. Alternatively, an external spherical image capturing unit may be attached to a general-purpose digital camera or smartphone to implement an image capturing device having substantially the same function as that of the spherical image capturing device 600.

The spherical image capturing device 600 illustrated in FIG. 4 includes an imaging unit 601, an image processor 604, an imaging controller 605, a microphone 608, an audio processor 609, a CPU 611, a ROM 612, a static random-access memory (SRAM) 613, a dynamic random-access memory (DRAM) 614, an operation device 615, an external device connection I/F 616, a communication device 617, an antenna 617a, and an acceleration and orientation sensor 618.

The imaging unit 601 includes two wide-angle lenses (so-called fisheye lenses) 602a and 602b, each having an angle of view equal to or greater than 180 degrees to form a hemispherical image, and two image sensors 603a and 603b corresponding to the wide-angle lenses 602a and 602b, respectively.

Each of the image sensors 603a and 603b includes an image sensor such as a CMOS sensor or a CCD sensor for converting an optical image formed by the corresponding wide-angle lens 602 (the wide-angle lens 602a or 602b) into an electric signal and outputting image data, a timing generation circuit for generating horizontal or vertical synchronization signals and pixel clocks for the image sensor, and a group of registers in which various commands or parameters are set for the operation of the imaging sensor 603a or 603b.

Each of the image sensors 603a and 603b of the imaging unit 601 is connected to the image processor 604 through a parallel I/F bus.

The image sensors 603a and 603b are connected to the imaging controller 605 through a serial I/F bus such as an I2C bus.

The image processor 604, the imaging controller 605, and the audio processor 609 are connected to the CPU 611 through a bus 610.

The ROM 612, the SRAM 613, the DRAM 614, the operation device 615, the external device connection I/F 616, the communication device 617, and the acceleration and orientation sensor 618 are also connected to the bus 610.

The image processor 604 acquires image data output from the image sensors 603a and 603b through the parallel I/F buses and performs predetermined processing on the image data. The image processor 604 combines the processed image data to generate data of an equirectangular projection image.

The imaging controller 605 functions as a master device while each of the image sensors 603a and 603b functions as a slave device. The imaging controller 605 sets commands in the group of registers of each of the image sensors 603a and 603b through the I2C bus.

The imaging controller 605 receives the commands from the CPU 611.

Further, the imaging controller 605 acquires status data of the group of registers of each of the image sensors 603a and 603b through the I2C bus. The imaging controller 605 transmits the acquired status data to the CPU 611.

The imaging controller 605 instructs the image sensors 603a and 603b to output the image data when a shutter button of the operation device 615 is pressed.

In some cases, the spherical image capturing device 600 displays a preview image or a moving image on a display (e.g., a display of a smartphone).

In the case of displaying a moving image, the image data is continuously output from the image sensors 603a and 603b at a predetermined frame rate (frames per minute).

As described later, the imaging controller 605 operates in cooperation with the CPU 611 to function as synchronization control means that synchronizes the time when the image sensor 603a outputs image data and the time when the image sensor 603b outputs the image data.

Although the spherical image capturing device 600 does not include a display in FIG. 4, the spherical image capturing device 600 may include a display.

The microphone 608 converts sound into audio data (signals).

The audio processor 609 acquires the audio data output from the microphone 608 through an I/F bus and performs predetermined processing on the audio data.

The CPU 611 controls the overall operation of the spherical image capturing device 600 and executes predetermined processing.

The ROM 612 stores various programs for execution by the CPU 611.

Each of the SRAM 613 and the DRAM 614 operates as a work memory to store programs to be executed by the CPU 611 or data currently processed by the CPU 611.

More specifically, in one example, the DRAM 614 stores image data currently processed by the image processor 604 and the data of the equirectangular projection image on which processing has been performed.

The operation device 615 collectively refers to various operation keys such as the shutter button.

The user operates the operation device 615 to input various image capturing modes or image capturing conditions.

The external device connection I/F 616 is an interface for connecting the spherical image capturing device 600 to various external devices, such as a USB flash drive or a PC.

The data of the equirectangular projection image, which is stored in the DRAM 614, may be stored in an external device such as an external medium through the external device connection I/F 616 or transmitted to an external device such as a smartphone through the external device connection I/F 616 as needed.

The communication device 617 communicates with communication terminals such as the smartphone 400 through the antenna 617a of the spherical image capturing device 600 by short-range wireless communication such as Wi-Fi, NFC, or BLUETOOTH.

The communication device 617 allows the spherical image capturing device 600 to transmit the data of the equirectangular projection image to a communication terminal such as the smartphone 400.

The acceleration and orientation sensor 618 calculates the orientation of the spherical image capturing device 600 from the Earth's magnetism to output orientation information.

The orientation information is an example of related information that is metadata described in compliance with Exif and is used for image processing such as image correction of the captured image.

The related information also includes the image capturing date and time, which indicates the date and time when the image is captured, and the data size of the image data.

The acceleration and orientation sensor 618 detects the change in tilt (roll, pitch, yaw) with the movement of the spherical image capturing device 600.

The change in tilt is one example of the related information (metadata) described in compliance with Exif and is used for image processing such as image correction of the captured image.

The acceleration and orientation sensor 618 further detects acceleration in three axial directions.

The spherical image capturing device 600 calculates the position (an angle with respect to the direction of gravity) of the spherical image capturing device 600, based on the acceleration detected by the acceleration and orientation sensor 618.

The acceleration and orientation sensor 618 allows the spherical image capturing device 600 to correct images with high accuracy.

### Overview

To facilitate understanding of the present embodiment, a description is given below of an example of the image distribution system 1 in which the subject user is a patient receiving home nursing care, the viewer is a student at a nursing school, and the distributor is a nurse who provides the home nursing care.

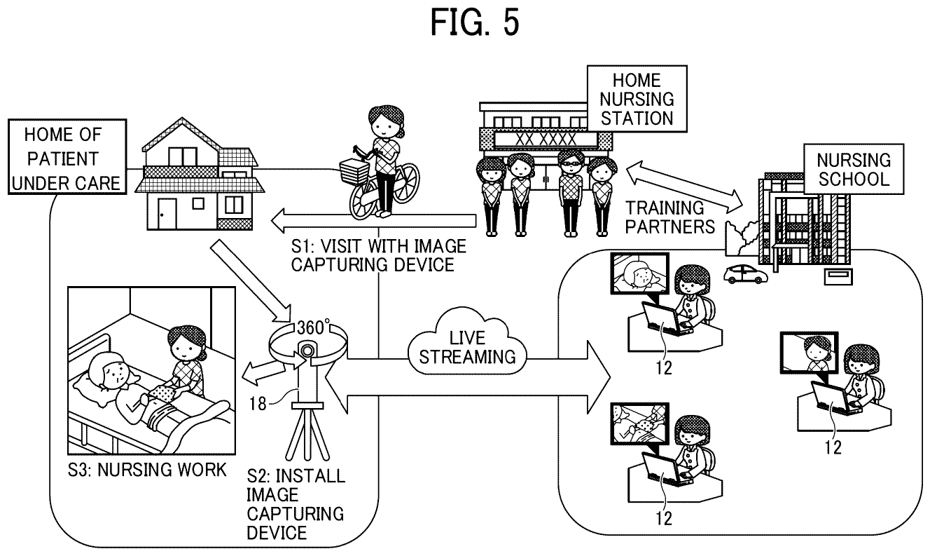

FIG. 5 is a diagram illustrating an overview of the image distribution system 1.

In FIG. 5, a home nursing station and a nursing school have a training partnership.

The home nursing station is a business office that provides home nursing services.

In step S1, the nurse as an example of the distributor visits the home of the patient under care with the image capturing device 18.

In step S2, the nurse installs the image capturing device 18 to capture images of the nursing work for the patient under care.

In step S3, the nurse starts capturing images with the image capturing device 18 and then performs the nursing work for the patient under care.

The image capturing device 18 captures the images of the nursing work performed by the nurse for the patient under care.

The image distribution device 10 manages the viewers who are permitted to view the images captured by the image capturing device 18. In other words, the image distribution device 10 controls the distribution destinations.

In FIG. 5, the students at the nursing school having a training partnership with the home nursing station are managed as the viewers who are permitted to view the images captured by the image capturing device 18.

The images captured by the image capturing device 18 are distributed and reproduced by live streaming to the first terminal devices 12 of the students at the nursing school managed by the image distribution device 10 as the viewers who are permitted to view the images.

When the images captured by the image capturing device 18 are 360-degree images, the students at the nursing school each operating the first terminal device 12 can view the images at a free angle for each student by operating the first terminal device 12 to select the angle of view.

The image distribution system 1 can control the distribution destination of the images to reflect the intention of the patient under care when the patient under care as an example of the subject user controls the distribution destination of the images captured by the image capturing device 18 as described later.

Accordingly, the image distribution system 1 can prevent the private information of the patient under care from being viewed by a user who is not intended by the patient under care.

### Functional Configuration

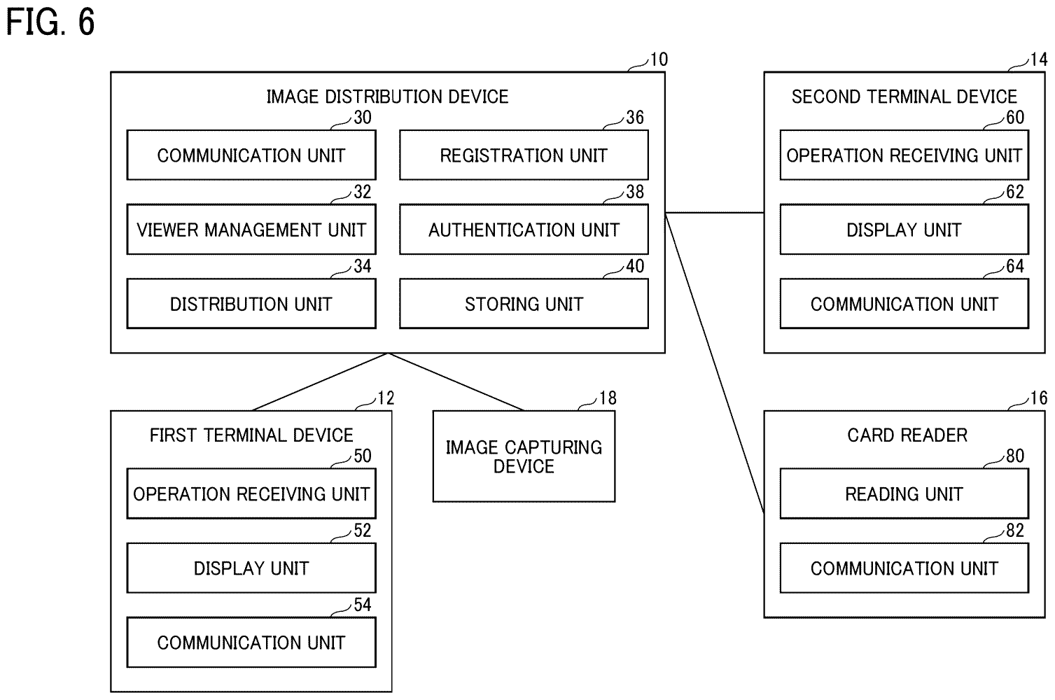

The image distribution system 1 is implemented by, for example, the functional configuration illustrated in FIG. 6.

FIG. 6 is a block diagram illustrating a functional configuration of the image distribution system 1.

In the functional configuration illustrated in FIG. 6, one or more functions unnecessary for the description of the present embodiment may be omitted for simplicity.

The image distribution device 10, the first terminal device 12, the second terminal device 14, and the card reader 16 of the image distribution system 1 execute an operating system (OS) and a program such as an application to implement, for example, the functional configuration illustrated in FIG. 6.

The image distribution device 10 illustrated in FIG. 6 includes a communication unit 30, a viewer management unit 32, a distribution unit 34, a registration unit 36, an authentication unit 38, and a storing unit 40.

The first terminal device 12 includes an operation receiving unit 50, a display unit 52, and a communication unit 54.

The second terminal device 14 includes an operation receiving unit 60, a display unit 62, and a communication unit 64.

The card reader 16 includes a reading unit 80 and a communication unit 82.

The operation receiving unit 50 of the first terminal device 12 receives various operations from the viewer such as a student at the nursing school.

The display unit 52 displays various types of information for the viewer.

The display unit 52 also displays the images captured by the image capturing device 18.

The communication unit 54 communicates with the image distribution device 10.

The operation receiving unit 60 of the second terminal device 14 receives various operations from the subject user such as a patient under care.

The display unit 62 displays various types of information for the subject user.

The communication unit 64 communicates with the image distribution device 10.

The reading unit 80 of the card reader 16 reads information from a recording medium such as a magnetic card or an IC card.

The recording medium such as a magnetic card or an IC card may be a recording medium associated with the information on a viewer who is permitted to view images by the subject user.

For example, a patient registration card of the subject user is an example of a recording medium associated with the information on the personnel of the hospital used by the subject user.

The Individual Number Card of the subject user is an example of a recording medium associated with the information on a family member set as a viewer by the subject user.

The communication unit 30 of the image distribution device 10 communicates with the first terminal device 12, the second terminal device 14, the card reader 16, and the image capturing device 18.

The viewer management unit 32 manages the viewers who are permitted to view the images captured by the image capturing device 18 as described later.

The distribution unit 34 distributes the images captured by the image capturing device 18 to the first terminal device 12 of the viewer managed by the viewer management unit 32.

In response to an action of the subject user to register a viewer, the registration unit 36 registers the viewer as a viewer managed by the viewer management unit 32.

The action of the subject user to register the viewer is an instruction from the subject user through manual input, voice input, or line-of-sight input for setting the viewer from the second terminal device 14.

The action of the subject user to register the viewer is an instruction to read information from the recording medium associated with the information on the viewer.

The authentication unit 38 authenticates a user who operates the first terminal device 12 or the second terminal device 14.

The storing unit 40 stores information necessary for the processing of the image distribution device 10, such as information or data of the subject user, the viewers, and the viewer candidates.

The functional configuration illustrated in FIG. 6 is merely an example. The first terminal device 12, the second terminal device 14, the card reader 16, or the image capturing device 18 may have some of the functions of the image distribution device 10.

The configuration of the image distribution device 10 illustrated in FIG. 6 is merely an example and may be divided into two or more devices.

### Processes

A description is given below of some processes of the image distribution system 1.

### Viewer Setting

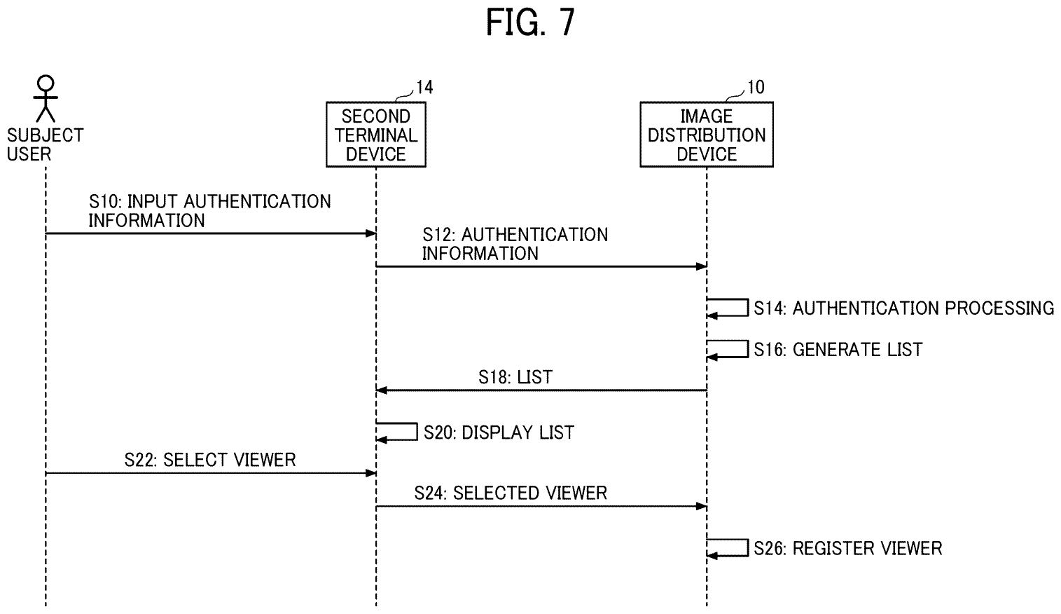

FIG. 7 is a sequence diagram illustrating a process of setting viewers in the image distribution system 1.

In step S10, the subject user operates the operation receiving unit 60 of the second terminal device 14 to activate a viewing management application and input the authentication information of the subject user.

In step S12, the communication unit 64 of the second terminal device 14 notifies the image distribution device 10 of the input the authentication information of the subject user.

In step S14, the authentication unit 38 of the image distribution device 10 performs authentication processing with the notified authentication information.

When the authentication is successful, in step S16, the authentication unit 38 generates a list.

By contrast, when the authentication is not successful, the authentication unit 38 ends the process of the sequence diagram illustrated in FIG. 7.

The description will be continued assuming that the authentication is successful.

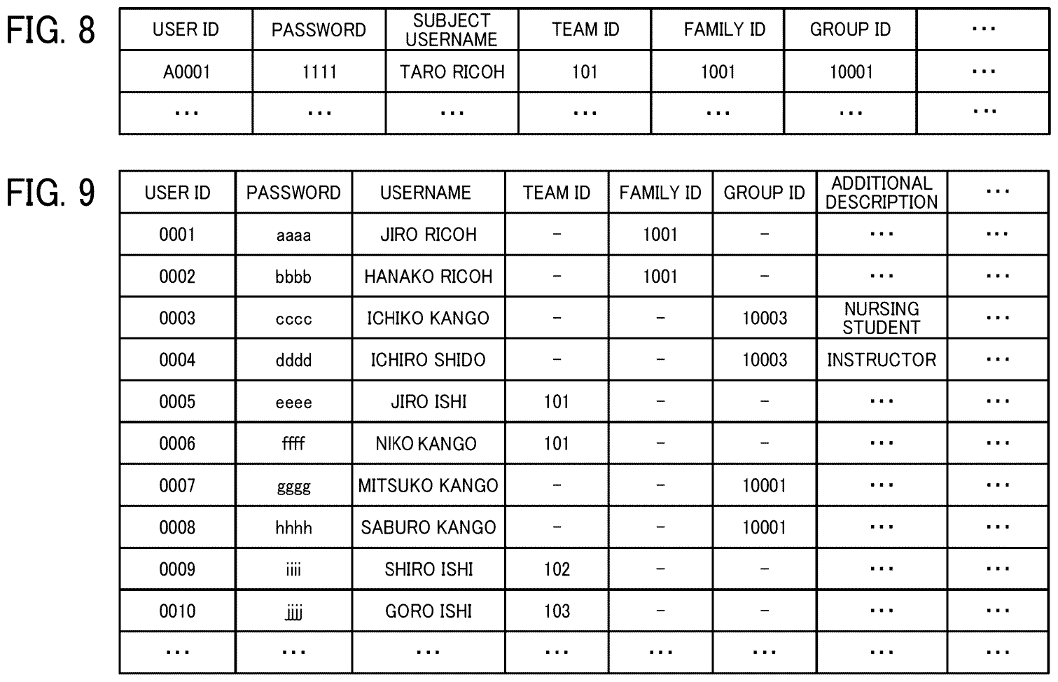

In step S16, the registration unit 36 generates the list to be presented to the subject user from, for example, the viewer candidate data illustrated in FIG. 9 with, for example, the information illustrated in FIG. 8 of the subject user who has succeeded in authentication as a key.

FIG. 8 is a table of information on subject users.

The information on the subject user includes a user ID, a password, a subject username, a team ID, a family ID, and a group ID as items.

The information on the subject user illustrated in FIG. 8 includes the items of the team ID, the family ID, and the group ID related to the subject user.

The user ID and the password are examples of authentication information of the subject user.

The subject username indicates the name of the subject user.

The team ID, the family ID, and the group ID are examples of keys used to select a default viewer or a viewer candidate to be presented to the subject user from, for example, the viewer candidate data illustrated in FIG. 9.

The default viewer is permitted to view the images on a screen of the viewing management application described later. Thus, check box control for selecting permission or non-permission of viewing is unavailable.

The check-box-controllable viewer candidate can be subjected to the check box control for determining whether to allow or disallow viewing on the screen of the viewing management application described later.

The team ID is an example of information for identifying, for example, personnel of a hospital in which the subject user is hospitalized.

The family ID is an example of information for identifying, for example, the family of the subject user.

The group ID is an example of information for identifying, for example, personnel of a business operator providing the subject user with home nursing services or personnel of a nursing care facility providing the subject user with nursing services.

The group ID is an example of information for identifying, for example, personnel or students at a nursing school or a nursing care school.

FIG. 9 is a table of viewer candidate data.

The viewer candidate data indicates the information on viewer candidates who can be viewers.

The viewer candidate data illustrated in FIG. 9 includes a user ID, a password, a username, a team ID, a family ID, a group ID, and an additional description as items.

The user ID and the password are examples of authentication information of the viewer candidate.

The username indicates the name of the viewer candidate.

The team ID is an example of information for identifying, for example, a hospital to which the viewer candidate belongs.

The family ID is an example of information for identifying, for example, the family of the viewer candidate.

The group ID is an example of information for identifying, for example, a business operator, a nursing facility, a nursing school, or a nursing care school to which the viewer candidate belongs.

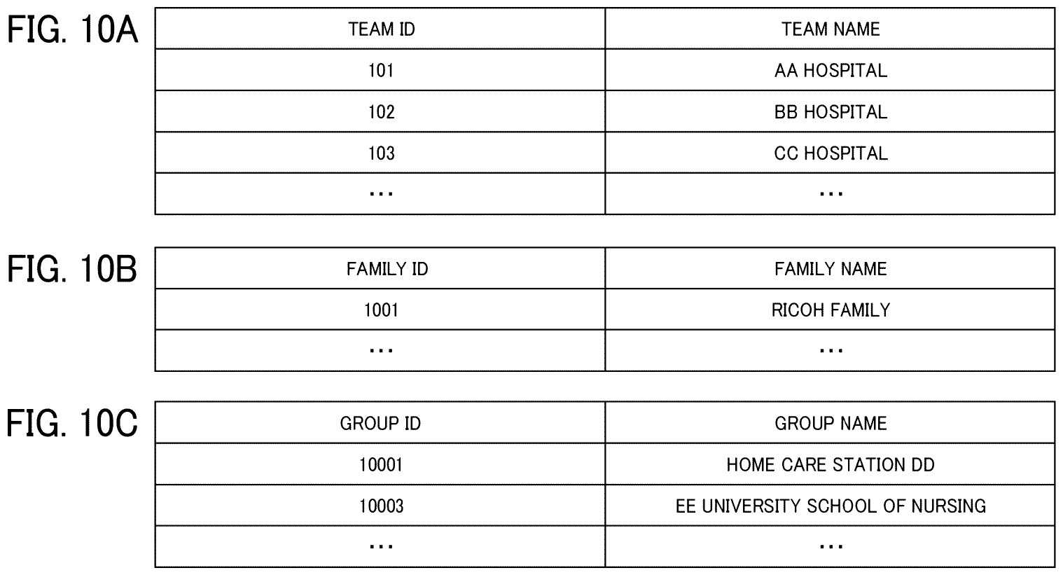

The team ID, the family ID, and the group ID are associated with, for example, the tables illustrated in FIGS. 10A, 10B, and 10C, respectively.

Specifically, FIG. 10A is a team table associated with the team ID.

FIG. 10B is a family table associated with the family ID.

FIG. 10C is a group table associated with the group ID.

For example, in the case of the information on the subject user illustrated in FIG. 8 and the viewer candidate data in FIG. 9, the registration unit 36 searches the viewer candidate data illustrated in FIG. 9 with the family ID “1001” in the information on the subject user as a key and selects the viewer candidates of the user IDs “0001” and “0002” as the default viewers.

When the registration unit 36 searches the viewer candidate data illustrated in FIG. 9 with the team ID “101” in the information on the subject user as a key, the registration unit 36 selects the viewer candidates of the user IDs “0005” and “0006” as the default viewers.

When the registration unit 36 searches the viewer candidate data illustrated in FIG. 9 with the group ID “10001” in the information on the subject user as a key, the registration unit 36 selects the viewer candidates of the user IDs “0007” and “0008” as the default viewers.

The registration unit 36 can search the viewer candidate data illustrated in FIG. 9 with the values of the items of the team ID, the family ID, and the group ID in the information on the subject user as keys and set the selected viewer candidates as the default viewers.

The list generated in step S16 includes the information on the default viewers and the check-box-controllable viewer candidates.

In step S18, the communication unit 30 of the image distribution device 10 responds to the second terminal device 14 with the list generated in step S16.

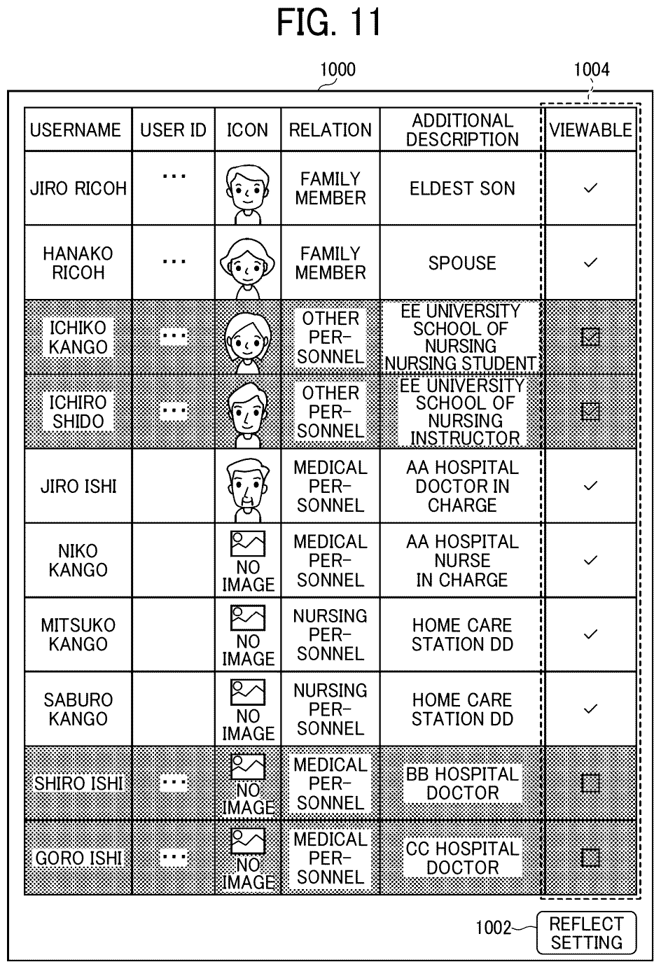

In step S20, the display unit 62 of the second terminal device 14 displays, for example, a screen 1000 of the viewing management application illustrated in FIG. 11.

FIG. 11 is an image diagram illustrating the screen 1000.

The display unit 62 uses the list received from the image distribution device 10 to display the screen 1000 of the viewing management application illustrated in FIG. 11.

On the screen 1000 of the viewing management application illustrated in FIG. 11, the default viewers included in the list are checked off in a check field 1004 by default and permitted to view the images.

The check box control is unavailable in the check field 1004 for the default viewers. Thus, viewing is not disallowed for the default viewers.

For the check-box-controllable viewer candidates included in the list, the check box control for determining whether to allow or disallow viewing is available in the check field 1004 on the screen 1000 of the viewing management application illustrated in FIG. 11.

In step S22, the subject user checks off the box of the viewer to be permitted to view the images in addition to the default viewer in the check field 1004 on the screen 1000 of the viewing management application illustrated in FIG. 11 displayed on the second terminal device 14, and then presses a setting reflection key 1002 to select viewers.

For example, on the screen 1000 of the viewing management application in FIG. 11, the subject user checks off the boxes of the nursing student and the instructor in the check field 1004 to permit the nursing student and the instructor to view the images to cooperate in the nursing practice.

In step S24, the communication unit 64 of the second terminal device 14 notifies the image distribution device 10 of the viewers set on the screen 1000 of the viewing management application illustrated in FIG. 11.

In response to the notification of the viewers set on the screen 1000 of the viewing management application illustrated in FIG. 11, in step S26, the registration unit 36 of the image distribution device 10 registers the viewers notified from the second terminal device 14.

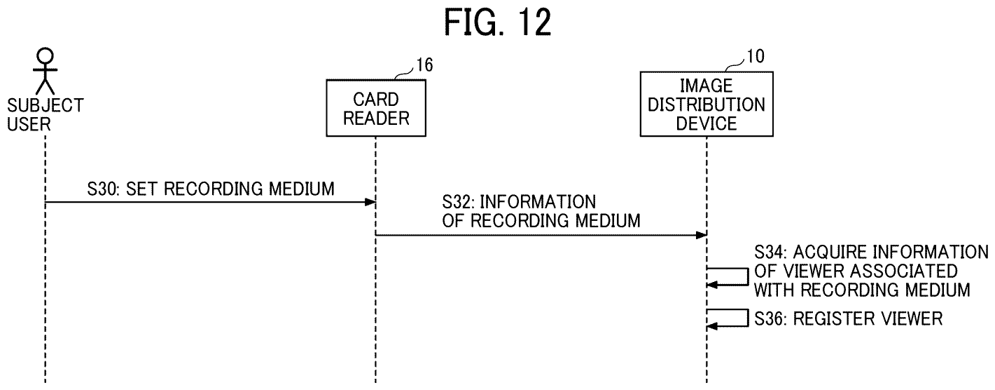

The process of setting viewers in the image distribution system 1 may be performed as illustrated in FIG. 12, for example.

FIG. 12 is a sequence diagram illustrating a process of setting viewers in the image distribution system 1.

In step S30, the subject user sets on or brings to the card reader 16 a recording medium such as an Individual Number Card or a patient registration card associated with the information on the viewer to be permitted to view the images.

The reading unit 80 of the card reader 16 reads the information from the recording medium such as an Individual Number Card or a patient registration card.

In step S32, the communication unit 82 of the card reader 16 notifies the image distribution device 10 of the information read by the reading unit 80.

In step S34, the viewer management unit 32 of the image distribution device 10 acquires the information on the viewer associated with the recording medium, based on the information in the recording medium notified from the card reader 16.

For example, when the information is read from the patient registration card of the hospital in which the subject user is hospitalized, the viewer management unit 32 acquires the information on the personnel of the hospital as the information on the viewer to be permitted to view the images.

When the information is read from the Individual Number Card of the subject user, the viewer management unit 32 may acquire the information on a family member of the subject user as the information on the viewer to be permitted to view the images.

In step S36, the registration unit 36 of the image distribution device 10 registers the viewer based on the information on the viewer acquired in step S34.

According to the process of the sequence diagram illustrated in FIG. 12, the image distribution device 10 can register a viewer based on the information on the viewer associated with a recording medium which is associated with the information on the viewer and from which the information is read.

Although the viewers are set in response to the action of the subject user in the processes of the sequence diagrams illustrated in FIGS. 7 and 12, a viewer may be set in response to a request from the viewer.

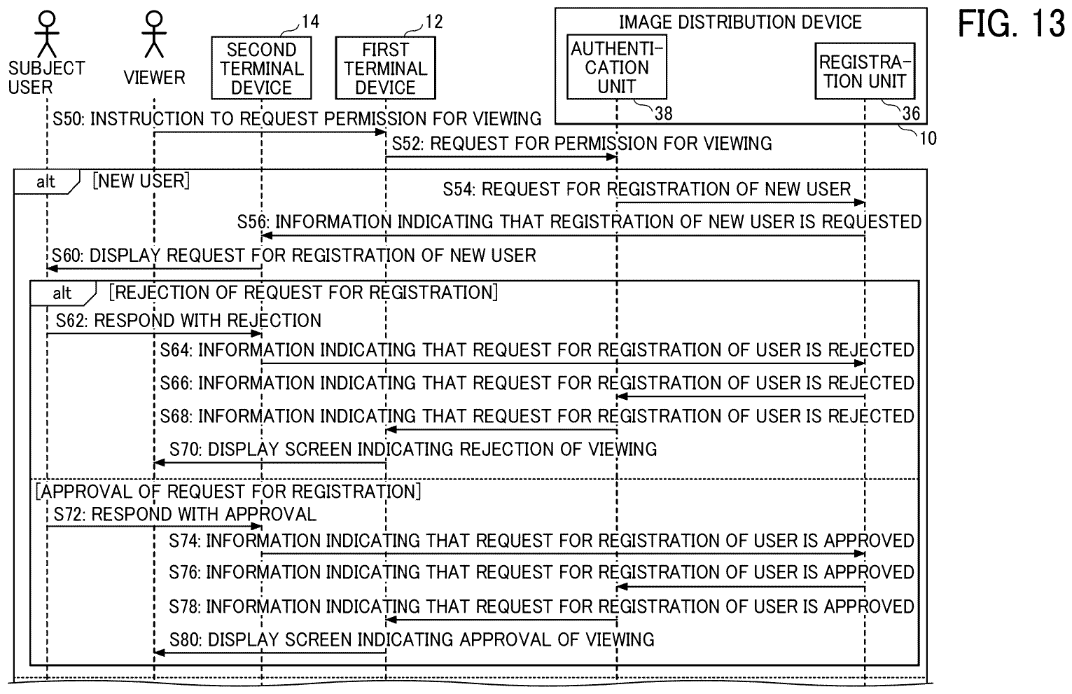

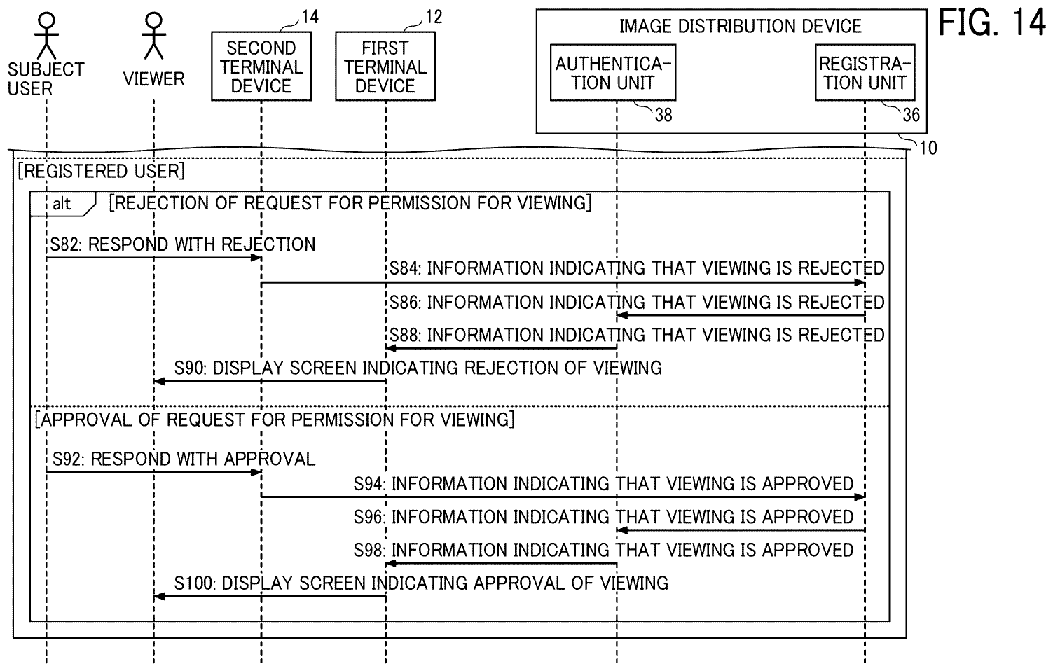

FIGS. 13 and 14 are sequence diagrams each illustrating a process of setting a viewer in response to a request from the viewer in the image distribution system 1.

In step S50, the viewer operates the operation receiving unit 50 of the first terminal device 12 to activate a viewing application, input the authentication information of the viewer, and request permission to view the captured images of the subject user.

In step S52, the communication unit 54 of the first terminal device 12 notifies the image distribution device 10 of the request for permission to view the images.

When the viewer who has requested permission to view the images in step S52 is a new user not managed by the viewer management unit 32, the image distribution device 10 proceeds to step S54.

In step S54, the authentication unit 38 requests the registration unit 36 to register a new user.

In step S56, the registration unit 36 notifies the second terminal device 14 that registration of a new user is requested.

In step S60, the display unit 62 of the second terminal device 14 displays the request for registration of a new user.

The request for registration of a new user displayed on the display unit 62 allows the subject user to recognize the request for permission to view the images from the new user.

Steps S62 to S70 are taken when the subject user responds with rejection to the notified request for permission to view the images.

In step S62, the subject user operates the operation receiving unit 60 of the second terminal device 14 to respond with a rejection of viewing.

In step S64, the communication unit 64 of the second terminal device 14 notifies the image distribution device 10 that the request for registration of the user is rejected.

In step S66, the registration unit 36 of the image distribution device 10 notifies the authentication unit 38 of the rejection of the request for registration of the user notified in step S64.

In step S68, the authentication unit 38 notifies the first terminal device 12 that the request for registration of the user is rejected.

In step S70, the display unit 52 of the first terminal device 12 displays a screen indicating rejection of viewing.

The user who has requested permission to view the images in step S50 can recognize that the request for permission to view the images is rejected by the subject user.

Steps S72 to S80 are taken when the subject user responds with approval to the notified request for permission to view the images.

In step S72, the subject user operates the operation receiving unit 60 of the second terminal device 14 to respond with an approval of viewing.

In step S74, the communication unit 64 of the second terminal device 14 notifies the image distribution device 10 of the request for registration of the user is approved.

In step S76, the registration unit 36 of the image distribution device 10 notifies the authentication unit 38 of the approval of the request for registration of the user notified in step S74.

In step S78, the authentication unit 38 notifies the first terminal device 12 that the request for registration of the user is approved.

In step S80, the display unit 52 of the first terminal device 12 displays a screen indicating approval of viewing.

The user who has requested permission to view the images in step S50 can recognize that the request for permission to view the images is approved by the subject user.

When the viewer who has requested permission to view the images in step S52 is a registered user managed by the viewer management unit 32, the image distribution device 10 notifies the second terminal device 14 of the request for permission to view the images from the registered user and causes the second terminal device 14 to display the request for permission to view the images from the registered user.

The request for permission to view the images from the registered user displayed on the display unit 62 of the second terminal device 14 allows the subject user to recognize the request for permission to view the images from the registered user.

Steps S82 to S90 are taken when the subject user responds with rejection to the notified request for permission to view the images.

In step S82, the subject user operates the operation receiving unit 60 of the second terminal device 14 to respond with a rejection of viewing.

In step S84, the communication unit 64 of the second terminal device 14 notifies the image distribution device 10 that the user's viewing is rejected.

In step S86, the registration unit 36 of the image distribution device 10 notifies the authentication unit 38 of the rejection of the user's viewing notified in step S84.

In step S88, the authentication unit 38 notifies the first terminal device 12 that the user's viewing is rejected.

In step S90, the display unit 52 of the first terminal device 12 displays the screen indicating rejection of viewing.

The user who has requested permission to view the images in step S50 can recognize that the request for permission to view the images is rejected by the subject user.

Steps S92 to S100 are taken when the subject user responds with approval to the notified request for permission to view the images.

In step S92, the subject user operates the operation receiving unit 60 of the second terminal device 14 to respond with an approval of viewing.

In step S94, the communication unit 64 of the second terminal device 14 notifies the image distribution device 10 that the user's viewing is approved.

In step S96, the registration unit 36 of the image distribution device 10 notifies the authentication unit 38 of the approval of the user's viewing notified in step S94.

In step S98, the authentication unit 38 notifies the first terminal device 12 that the user's viewing is approved.

In step S100, the display unit 52 of the first terminal device 12 displays the screen indicating approval of viewing.

The user who has requested permission to view the images in step S50 can recognize that the request for permission to view the images is approved by the subject user.

### Distribution of Captured Images of Subject User

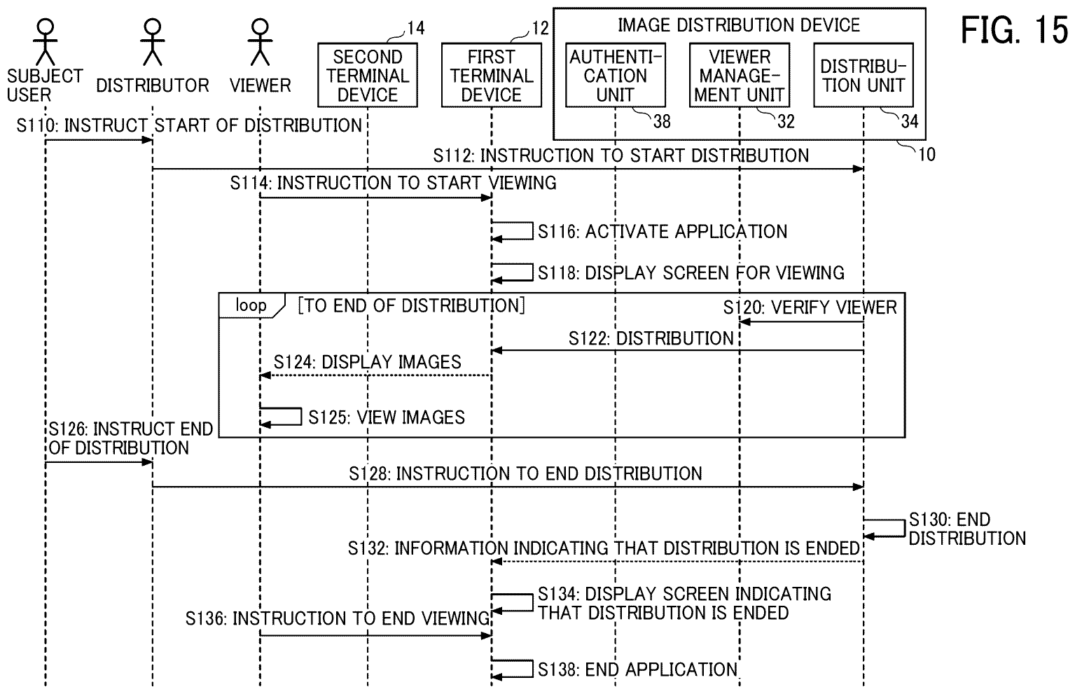

FIG. 15 is a sequence diagram illustrating a process of controlling a distribution destination of captured images of a subject user to reflect the intention of the subject user in the image distribution system 1.

Before performing the process of the sequence diagram illustrated in FIG. 15, the subject user sets the viewer who is permitted to receive the captured images of the subject user as illustrated in FIG. 7, for example.

During the process of the sequence diagram illustrated in FIG. 15, the subject user may set the viewer who is permitted to receive the captured images of the subject user as illustrated in FIG. 13 or 14, for example.

After the distributor installs the image capturing device 18, in step S110, the subject user instructs the distributor to start distribution.

In step S112, the distributor operates the image distribution device 10 to start the distribution of the images.

In step S114, the viewer operates the operation receiving unit 50 of the first terminal device 12 to start viewing the images.

In step S116, the first terminal device 12 activates the viewing application.

In step S118, the first terminal device 12 displays a screen for viewing on the display unit 52.

A series of steps S120, S122, and S124 is repeated to the end of the distribution of the images captured by the image capturing device 18.

In step S120, the distribution unit 34 of the image distribution device 10 verifies the information on the viewer who is permitted to view the images with the viewer management unit 32.

In step S122, the distribution unit 34 distributes the images captured by the image capturing device 18 to the first terminal device 12 of the viewer who is permitted to view the images.

In step S124, the display unit 52 of the first terminal device 12 displays the images distributed from the image distribution device 10.

In step S125, the viewer who is permitted to view the images views the captured images of the subject user.

The image distribution system 1 controls the distribution destination of the images to be the viewer determined and allowed by the subject user to view the images. Thus, the image distribution system 1 controls the distribution destination of the images to reflect the intention of the subject user.

In step S126, the subject user instructs the distributor to end distribution.

In step S128, the distributor operates the image distribution device 10 to end the distribution of the images.

In step S130, the distribution unit 34 of the image distribution device 10 ends distribution.

In step S132, the distribution unit 34 notifies the first terminal device 12 that the distribution is ended.

In step S134, the first terminal device 12 displays, on the display unit 52, a screen indicating that the distribution has ended.

In step S136, the viewer operates the operation receiving unit 50 of the first terminal device 12 to end viewing of the images.

In step S138, the first terminal device 12 ends the viewing application.

As described above, the image distribution system 1 controls the distribution destination of the images of the subject user to reflect the intention of the subject user, thereby distributing the private information of the subject user to an appropriate range and preventing the private information from being distributed to an unintended user.

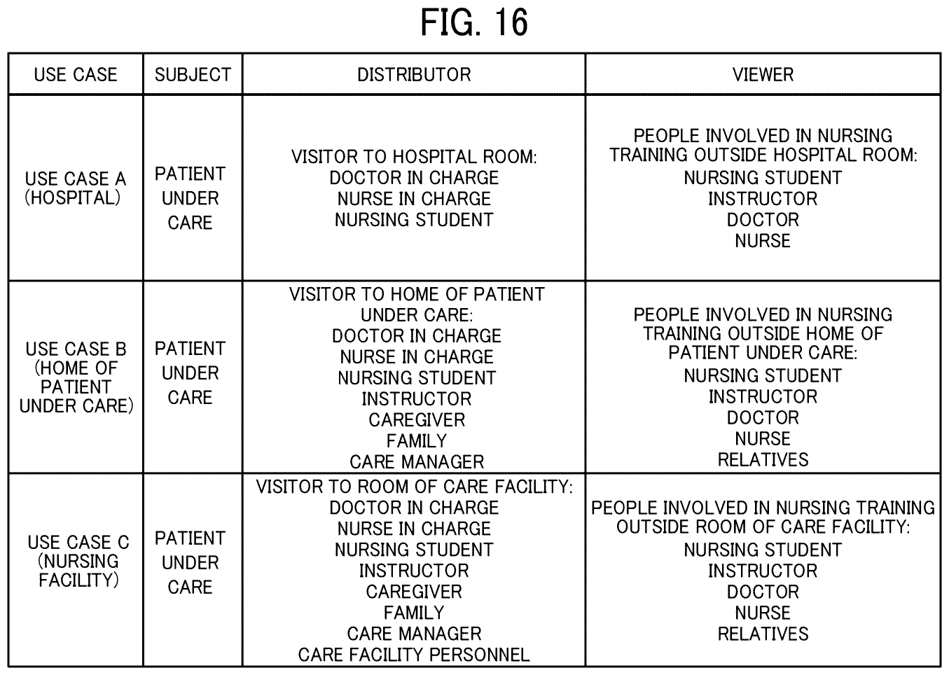

The image distribution system 1 can be used in, for example, a use case as illustrated in FIG. 16.

FIG. 16 is a table of se cases of the image distribution system 1.

Use case A illustrates a subject, a distributor, and a viewer in a case where a nursing practice performed at a hospital is used.

Use case B illustrates a subject, a distributor, and a viewer in a case where a nursing practice performed at the home of the patient under care is used.

Use case C illustrates a subject, a distributor, and a viewer in a case where a nursing practice performed at a nursing facility is used.

### Provision of Photographic Image of Viewer

When photographs of the default viewers and the check-box-controllable viewer candidates included in the list have been registered, the photographs are displayed as icons on the screen 1000 of the viewing management application in FIG. 11.

By contrast, when the photographs of the default viewers and the check-box-controllable viewer candidates included in the list have not been registered, a message (e.g., “NO IMAGE”) indicating that the photograph is not registered is displayed.

When the subject user determines whether to allow or disallow a viewer to view the images, it is desirable that the photographs of the check-box-controllable viewer candidates are registered because the photographs may help the subject user to determine whether to allow or disallow the viewer to view the images.

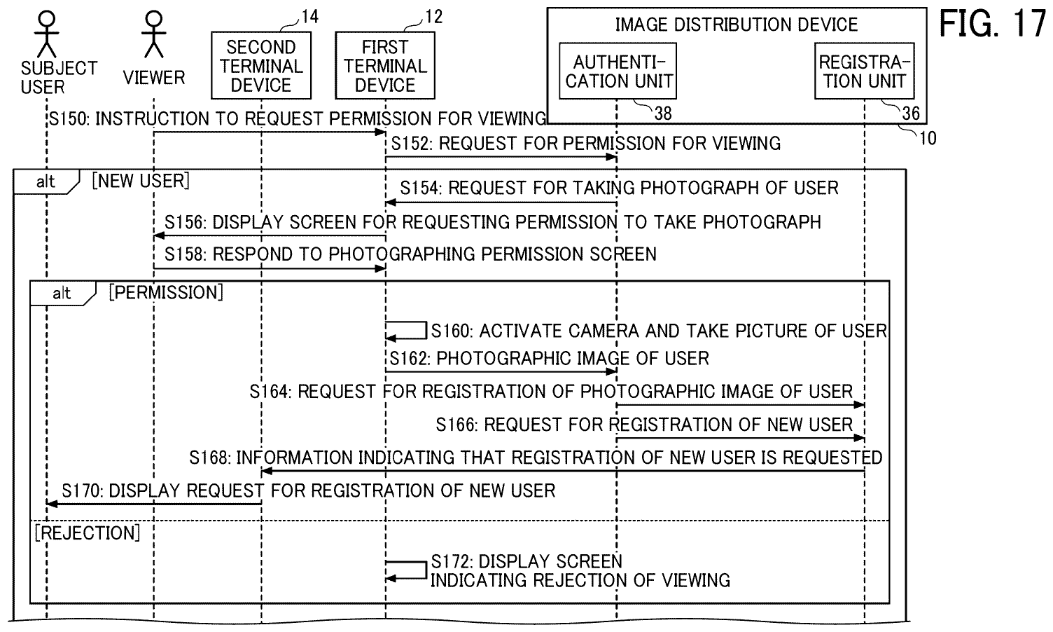

For this reason, in the image distribution system 1, a viewer to be set may be requested to take a photograph as illustrated in FIG. 17, in the processes of the sequence diagrams illustrated in FIGS. 13 and 14.

FIG. 17 is a sequence diagram illustrating a process of requesting a viewer to be set to take a photograph.

In step S150, the viewer operates the operation receiving unit 50 of the first terminal device 12 to activate the viewing application, input the authentication information of the viewer, and request permission to view the captured images of the subject user.

In step S152, the communication unit 54 of the first terminal device 12 notifies the image distribution device 10 of the request for permission to view the images.

When the viewer who has requested permission to view the images is a new user not managed by the viewer management unit 32, the image distribution device 10 proceeds to step S154.

In step S154, the authentication unit 38 requests the first terminal device 12 to take a photograph of the viewer.

In step S156, the first terminal device 12 displays, on the display unit 52, a photographing permission screen for requesting permission to take a photograph.

In step S158, the viewer operates the first terminal device 12 to provide a response (approval or rejection) on the photographing permission screen.

Steps S160 to S170 are taken when the response provided on the photographing permission screen is approval.

In step S160, the first terminal device 12 activates a camera to take a photograph of the viewer.

In step S162, the communication unit 54 of the first terminal device 12 transmits a photographic image of the viewer to the image distribution device 10.

In step S164, the authentication unit 38 of the image distribution device 10 requests the registration unit 36 to register the photographic image of the viewer.

In step S166, the authentication unit 38 requests the registration unit 36 to register a new user.

In step S168, the registration unit 36 notifies the second terminal device 14 of the request for registration of a new user.

In step S170, the display unit 62 of the second terminal device 14 displays the request for registration of a new user.

The request for registration of a new user displayed on the display unit 62 allows the subject user to recognize the request for permission to view the images from the new user.

Step S172 is taken when the response provided on the photographing permission screen is rejection.

In step S172, the display unit 52 of the first terminal device 12 displays the screen indicating rejection of viewing.

The viewer who has rejected the request in responding to the photographing permission screen in step S158 can recognize that the request for permission to view the images is rejected by the subject user.

Steps as in steps S62 to S70 or steps S72 to S80 illustrated in FIG. 13 are taken after step S170, and thus the description thereof will be omitted.

When the viewer who has requested permission to view the images in step S52 is a registered user managed by the viewer management unit 32, steps as in steps S82 to S90 or steps S92 to S100 illustrated in FIG. 14 are taken, and thus the description thereof will be omitted.

According to one or more aspects, an image distribution system controls a distribution destination of captured images of a subject user to reflect the intention of the subject user.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention.

Any one of the above-described operations may be performed in various other ways, for example, in an order different from the one described above.

The functionality of the elements disclosed herein may be implemented using circuitry or processing circuitry which includes general purpose processors, special purpose processors, integrated circuits, application-specific integrated circuits (ASICs), field-programmable gate arrays (FPGAs), and/or combinations thereof which are configured or programmed, using one or more programs stored in one or more memories, to perform the disclosed functionality. Processors are considered processing circuitry or circuitry as they include transistors and other circuitry therein. In the disclosure, the circuitry, units, or means are hardware that carry out or are programmed to perform the recited functionality. The hardware may be any hardware disclosed herein which is programmed or configured to carry out the recited functionality.

There is a memory that stores a computer program which includes computer instructions. These computer instructions provide the logic and routines that enable the hardware (e.g., processing circuitry or circuitry) to perform the method disclosed herein. This computer program can be implemented in known formats as a computer-readable storage medium, a computer program product, a memory device, a record medium such as a compact disc-read-only memory (CD-ROM) or digital versatile disk (DVD), and/or the memory of an FPGA or ASIC.

The apparatuses or devices described in the above-described embodiments are merely one example of multiple computing environments that implement the embodiments disclosed herein.

The present invention, however, is not limited to the above-described embodiments, and constituent elements of the above-described embodiments include elements that may be easily conceived by those skilled in the art, substantially the same elements, and elements within so-called equivalent ranges.

Various omissions, substitutions, changes, and combinations of the constituent elements may be made without departing from the gist of the above-described embodiments.

1. An image distribution system, comprising:
a first terminal device operated by a viewer; and
an information processing device to control distribution of a captured image of a subject user to the first terminal device,
the information processing device including circuitry configured to:
manage information on the viewer permitted to view the captured image of the subject user;
distribute the captured image of the subject user to the first terminal device of the managed viewer; and
register the viewer as the managed viewer in response to an action of the subject user to register the viewer,
the first terminal device including a display to display the distributed captured image of the subject user.

2. The image distribution system according to claim 1,
wherein the circuitry is configured to receive, as the action of the subject user to register the viewer, an instruction to register the viewer from a second terminal device operated by the subject user.

3. The image distribution system according to claim 1,
wherein the circuitry is configured to receive, as the action of the subject user to register the viewer, an instruction to register the viewer using a recording medium associated with the information on the viewer to be registered.

4. The image distribution system according to claim 1,
wherein the circuitry is configured to register, as the managed viewer, the viewer whose request for registration of the viewer is approved by the subject user.

5. The image distribution system according to claim 4,
wherein the circuitry is configured to request the viewer who has made the request for registration to take a photograph when the photograph of the viewer is not registered.

6. The image distribution system according to claim 2,
wherein the action of the subject user is an instruction from the subject user through manual input, voice input, or line-of-sight input to the second terminal device.

7. An image distribution method, comprising:
managing information on a viewer permitted to view a captured image of a subject user;
distributing the captured image of the subject user to a first terminal device of the managed viewer;
registering the viewer as the managed viewer in response to an action of the subject user to register the viewer; and
displaying the distributed captured image of the subject user.

8. An image distribution device, comprising circuitry configured to:
manage information on a viewer permitted to view a captured image of a subject user;
distribute the captured image of the subject user to a first terminal device of the managed viewer; and
register the viewer as the managed viewer in response to an action of the subject user to register the viewer.
